SEQUENCE LISTING (14 sequences)

SEQ ID NO: 1 - DNA, 63 bp, Homo sapiens
  misc_feature: CTGF = connective tissue growth factor
gcgccgcccggagcgtataaaagcctcggccgcccgccccaaactcacacaacaactcttccg

SEQ ID NO: 2 - DNA, 63 bp, Homo sapiens
  misc_feature: E1B = adenovirus E1B gene
gcggggcttaaagggtatataatgcgccgtgggctaatcttggttacatctgacctcatggag

SEQ ID NO: 3 - DNA, 63 bp, Homo sapiens
  misc_feature: AdML = adenovirus major late promoter
ttcctgaaggggggctataaaagggggtgggggcgcgttcgtcctcactctcttccgcatgaa

SEQ ID NO: 4 - DNA, 64 bp, Homo sapiens
  misc_feature: Comp C3 = complement C3
gggaaagcaggagccagataaaaagccagctccagcaggcgctgctcactcctccccatcctct

SEQ ID NO: 5 - DNA, 63 bp, Homo sapiens
  misc_feature: PAI-1 = plasminogen activator inhibitor-1
cctgcccacatctggtataaaaggaggcagtggcccacagaggagcacagctgtgtttggctg

SEQ ID NO: 6 - DNA, 63 bp, Homo sapiens
  misc_feature: SAA = serum amyloid A
gagaccagcaaggctcactataaatagcagccacctctccctggcagacagggacccgcagct

SEQ ID NO: 7 - DNA, 63 bp, Homo sapiens
  misc_feature: SOD2 = manganese superoxide dismutase
gctggggtcgcggccctgctcccggcgctttcttaaggcccgcgggcggcgcaggagcggcac

SEQ ID NO: 8 - DNA, 63 bp, Homo sapiens
  misc_feature: TK = thymidine kinase
tcccaggtccacttcgcatattaaggtgacgcgtgtggcctcgaacaccgagcgaccctgcag

SEQ ID NO: 9 - DNA, 63 bp, Homo sapiens
  misc_feature: Bax = Bax
actcagtttttagtcatctataacgtcctgcctggaagcatgctattttgggcctctgagctt

SEQ ID NO: 10 - DNA, 64 bp, Homo sapiens
  misc_feature: Cyclin D1 = Cyclin D1
gactacaggggagttttgttgaagttgcaaagtcctggagcctccagagggctgtcggcgcagt

SEQ ID NO: 11 - DNA, 63 bp, Homo sapiens
  misc_feature: Cyclin E = Cyclin E
ggagccgcggcggggcggtgcgagggcgggccggggccggttccgcgcgcagggattttaaat

SEQ ID NO: 12 - DNA, 63 bp, Homo sapiens
  misc_feature: p21 = p21
gcccgggcggggcggttgtatatcagggccgcgctgagctgcgccagctgaggtgtgagcagc

SEQ ID NO: 13 - DNA, 849 bp, Homo sapiens
  misc_feature: SAA promoter used
tggccaccatgctcctccataagcctctgcagagctaatctgaccctgttgatgttctcatgagagagtgatctgaatgccccctgaacccctccgtgataatacagcagaccaagagctctcccacccttccctgcctggatgctgggcacgtccccagctgggctgcctatttaacgcaccacactctcattctcccaaggtggggctccaggactaggctggggcagcagaaagtccccctctctacattgtccttggctcaggagccaacttagaaaaagcatttccaaattggctaagccagcggagcagagattttctgtgctgagaaatatcaggacatccagaggggtggaaggaggcttccagggcacacatgagatgtggcaggggtaggctgtccgttttaaagcttaaagctttagacatgaactcacagggacttcagtcagggtcatctgccatgtggcccagcagggcccatcctgaggaaatgaccggtatagtcaggagctggctgaagagctgccctcactccacaccttccagcagcccaggtgccgccatcacggggctcccactggcatctctgcagctgcacttcccccaatgctgaggagcagagctgatctagcaccctgtccattgccaaggcacagcaaacctctcttgttcccataggttacacaactgggataaatgacccgggatgaagaaaccaccggcatccaggaacttgtcttagaccagtttgtaggggaaatgacctgcagggactttccccagggaccacatccagcttttcttccctcccaagagaccagcaaggctcactataaatagcag

SEQ ID NO: 14 - DNA, 849 bp, Homo sapiens
  misc_feature: TATA mutant SAA promoter used
tggccaccatgctcctccataagcctctgcagagctaatctgaccctgttgatgttctcatgagagagtgatctgaatgccccctgaacccctccgtgataatacagcagaccaagagctctcccacccttccctgcctggatgctgggcacgtccccagctgggctgcctatttaacgcaccacactctcattctcccaaggtggggctccaggactaggctggggcagcagaaagtccccctctctacattgtccttggctcaggagccaacttagaaaaagcatttccaaattggctaagccagcggagcagagattttctgtgctgagaaatatcaggacatccagaggggtggaaggaggcttccagggcacacatgagatgtggcaggggtaggctgtccgttttaaagcttaaagctttagacatgaactcacagggacttcagtcagggtcatctgccatgtggcccagcagggcccatcctgaggaaatgaccggtatagtcaggagctggctgaagagctgccctcactccacaccttccagcagcccaggtgccgccatcacggggctcccactggcatctctgcagctgcacttcccccaatgctgaggagcagagctgatctagcaccctgtccattgccaaggcacagcaaacctctcttgttcccataggttacacaactgggataaatgacccgggatgaagaaaccaccggcatccaggaacttgtcttagaccagtttgtaggggaaatgacctgcagggactttccccagggaccacatccagcttttcttccctcccaagagaccagcaaggctatctataacgtgcag

(19) United States
(12) Patent Application Publication
(10) Pub. No.: US 2003/0186424 A1
(43) Pub. Date: Oct. 2, 2003

(54) Reagents and methods for identifying and modulating expression of genes regulated by CDK inhibitors

(76) Inventors: Igor B. Roninson, Wilmette, IL (US); Neil D. Perkins, Dundee (GB); David J. Gregory, Montreal (CA); Jason C. Poole, Chicago, IL (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/231,537

(22) Filed: Aug. 29, 2002

Related U.S. Application Data
(60) Provisional application No. 60/315,791, filed on Aug. 29, 2001.

(51) Int. Cl. A61K 39/23; A61K 39/235; C12N 7/00; C12N 7/01; C12N 7/02; C12N 15/00; C12N 15/09; C12N 15/63; C12N 15/70; C12N 15/74; C12N 5/00; C12N 5/02

(52) U.S. Cl. 435/235.1; 435/239; 435/325; 435/320.1; 424/233.1

(57) ABSTRACT

This invention provides methods and reagents for identifying compounds that inhibit the induction of genes involved in cancer, age-related diseases, and viral diseases, such genes being induced by p21^{Waf1/Cip1/Sdi1}.

This application claims priority to U.S. Provisional Application Serial No.: 60/315,791, filed Aug. 29, 2001.

This application was supported by a grant from the National Institutes of Health, Nos. R01 CA89636 and R01 AG17921. The government may have certain rights in this invention.

### BACKGROUND OF THE INVENTION

1. Field of the Invention

This invention is related to induction of cellular and viral gene expression in cells expressing a member of a class of cellular gene products termed cyclin dependent kinase (CDK) inhibitors, induced in cells in response to stress and at the onset of senescence. More specifically, the invention provides reagents and methods for identifying compounds that modulate changes in cellular gene expression mediated by the CDK inhibitor, p21^{Waf1/Cip1/Sdi1}, hereinafter referred to as p21. The invention provides reagents for identifying such compounds that are recombinant mammalian cells containing recombinant expression constructs encoding reporter genes operably linked to promoters from genes whose expression is induced by p21 Methods for using said compounds to inhibit p21-mediated cellular or viral gene expression induction, methods for treating or preventing viral disease, and methods for treating or preventing pathogenic consequences of senescence and aging mediated by p21-induced gene expression are also provided.

2. Summary of the Related Art

Cell cycle progression is regulated to a large extent by a set of serine/threonine kinases, known as cyclin-dependent kinases (CDKs). A special group of proteins, known as CDK inhibitors, interact with and inhibit CDKs, thus causing cell cycle arrest in a variety of physiological situations (see Sielecki et al., 2000, J. Med. Chem. 43:1-18 and references therein). Cellular expression levels of the most pleiotropic of the known CDK inhibitors, p21, are increased in response to a variety of stimuli, including DNA-damaging and differentiating agents. p21 induction is also a frequent corollary of infection with different viruses (Majumder et al., 2001, J. Virol. 75: 1401-7; Park et al., 2000, Oncogene 19: 3384; de La Fuente et al., 2000, J. Virol. 74: 7270; Schmidt-Grimminger et al. 1998, Am. J. Pathol. 152: 1015).

p21 induction in some cases is mediated through transcriptional activation of the p21 gene by p53, but p21 is also regulated by a variety of p53-independent factors (reviewed in Gartel & Tyner, 1999, Exp. Cell Res. 227: 171-181). While p21 is not a transcription factor per se, it has indirect effects on cellular gene expression that may play a role in its cellular functions (Dotto, 2000, BBA Rev. Cancer 1471:M43-M56 and references therein). One of the consequences of CDK inhibition by p21 is dephosphorylation of Rb, which in turn inhibits E2F transcription factors that regulate many genes involved in DNA replication and cell cycle progression (Nevins, 1998, Cell Growth Differ. 9: 585-593). A comparison of p21-expressing cells (p21 +/+) and p21-nonexpressing cells (p21 −/−) has implicated p21 in radiation-induced inhibition of several genes involved in cell cycle progression (de Toledo et al., 1998, Cell Growth Differ. 9: 887-896). An effect of p21 which is of special importance to the instant invention is stimulation of the transcription cofactor histone acetyltransferase p300, that enhances many inducible transcription factors including NFκB (Perkins et al., 1988, Science 275: 523-527). Activation of p300 may have a pleiotropic effect on gene expression (Snowden & Perkins, 1988, Biochem. Pharmacol. 55: 1947-1954). p21 may also affect gene expression through its interactions with many transcriptional regulators and coregulators other than CDK, such as JNK kinases, apoptosis signal-regulating kinase 1, Myc and others (Dotto, 2000, BBA Rev. Cancer 1471:M43-M56). These interactions may affect the expression of genes regulated by the corresponding pathways.

The transcriptional coactivators p300 and CREB binding protein (CBP) function as pleiotropic regulators of gene expression in mammalian cells (Goodman & Smolik, 2000, Genes Dev. 14: 1553-1577; Snowden & Perkins, 1998, Biochem. Pharmacol. 55: 1947-1954). p300 and CBP are recruited to promoters by a large number of DNA-binding proteins and can stimulate gene expression either through their inherent histone acetyl transferase (HAT) activity or through their ability to interact with other coactivators and components of the basal transcriptional machinery (Goodman & Smolik, 2000, ibid.). The requirement for p300/CBP HAT activity or other functions can vary at different promoters under different conditions (Gamble & Freedman, 2002, Trends Biochem. Sci. 27: 165-167). For example, in one study the HAT activity of CBP was found to be essential for transcription of a Retinoic Acid Receptor (RAR) dependent reporter, but dispensable for a CREB dependent reporter (Korzus et al., 1998, Science 279: 703-707). Similarly, despite being dependent on p300, MyoD driven reporter gene transcription and terminal cell cycle arrest, were found not to require its HAT activity (Puri et al., 1997, Mol. Cell 1: 35-45). Moreover, when activated by cAMP, Pit-1 dependent transcription was found to require the HAT activity of CBP. When activated by insulin, however, the HAT activity of PCAF was necessary instead, despite both coactivators being required in each case (Xu et al., 1998, Nature 395: 301-306). Therefore, p300/CBP should be considered as multifunctional proteins, which can act in different ways under different circumstances to specifically regulate transcription (Gamble & Freedman, 2002, ibid.).

Among their many functions, p300 and CBP are required for the function of transcription factors that regulate both cellular proliferation and growth arrest, such as E2F, c-Jun, p53, NF-κB and MyoD (Goodman & Smolik, 2000, ibid.; Snowden & Perkins, 1998, ibid.). Until recently, it was unclear whether p300 and CBP behave in a passive manner, merely being recruited to promoters and enhancers by these proteins and contributing in an unregulated fashion towards the process of gene activation, or whether they have a more dynamic regulatory function. Supporting this latter role, some of the present inventors recently demonstrated that transcriptional activation by p300 and CBP is regulated by p21 (Snowden et al., 2000, Mol. Cell. Biol. 20: 2676-2686). p21 strongly stimulates p300/CBP transactivation by inhibiting the function of a potent transcriptional repression domain, CRD1, present in both proteins (amino acids 1004-1044 in p300, 1019-1082 in CBP). CRD1 could regulate both full-length p300 as well as its amino and carboxy termini, although stronger induction by p21 was observed with constructs encoding amino terminal sequences (Snowden et al., 2000, ibid.). CRD1 functioned independently of p300/CBP HAT activity and in isolation was also capable of repressing transcription (Snowden et al., 2000, ibid). Deletion of CRD1, both within the context of full-length p300 and amino and carboxy terminal constructs, abolished p21 inducibility. Because the interaction of p300 and CBP with the DNA-binding proteins that recruit them is often a complex and highly regulated event, the majority of these studies were performed with Gal4 fusion proteins, which allowed the function of these coactivators to be studied in relative isolation. Importantly, it was also demonstrated that CRD1 could repress transcription of wild type p53 and a Gal4 fusion with the p53 transactivation domain when in the context of full length, non-Gal4 linked p300 (Snowden et al., 2000, ibid.). While other p21 dependent effects have generally been associated with transcriptional inhibition, this p300 CRD1 domain dependent mechanism is currently the only pathway described through which p21 can induce gene expression (Perkins, 2002, Cell Cycle 1: 39-41).

p21 expression from an inducible promoter was shown to produce multiple changes in cellular gene expression, with pronounced biological specificity. Most of the genes that are repressed upon p21 induction are involved in cell cycle progression. On the other hand, genes that are upregulated by p21 include a high fraction of secreted and transmembrane proteins that affect neighboring cells and tissues. Many of p21-inducible genes encode tumor-promoting secreted factors with mitogenic or anti-apoptotic activities, as well as genes implicated in age-related diseases, such as Alzheimer's disease, atherosclerosis, amyloidosis and arthritis (Chang et al., 2000, Proc. Natl. Acad. Sci. USA 97: 4291-4296; International Patent Application, Publication Nos. WO 00/61751; WO 01/38532 and WO 02/066681). Thus, there is a need in the art to identify compounds that will prevent the induction of gene expression by p21, as such compounds should be of therapeutic benefit for a variety of diseases. Induction of gene expression by p21 results from transcriptional stimulation, since functional promoters of all the tested p21-inducible genes were found to be stimulated by p21 (see International Patent Application, Publication No. WO 02/066681). The role of p300 and CBP as mediators of this transcriptional effect of p21 is of obvious interest. In view of the fact that there are a large number of transcription factors with which p300 and CBP interact but only a relatively small number of genes induced by p21, the mechanism through which p21 utilizes CRD1 function must be highly selective. Thus, there is a need in the art to identify regions and portions of promoters from p21-inducible genes as a way to discover compounds that inhibit or stimulate p21-mediated expression activation of these genes.

### SUMMARY OF THE INVENTION

The invention provides methods and reagents for identifying compounds that inhibit p21-mediated induction of cellular and viral gene transcription. The invention also provides compounds that inhibit p21-mediated induction of cellular and viral gene transcription, and methods for using said compounds to inhibit p21-mediated cellular or viral gene expression induction. Methods for treating or preventing viral disease, and methods for treating or preventing pathogenic consequences of senescence and aging mediated by p21-induced gene expression are also provided.

In a first aspect, the invention provides a mammalian cell in which p21 expression can be induced, the cell comprising two recombinant expression constructs: a first recombinant expression construct that encodes a fusion protein between a sequence-specific DNA-binding protein and p300 or CRB or a truncated version thereof that maintains transcription activation activity and comprises a CRD1 amino acid sequence motif; and a second recombinant expression construct encoding a reporter gene operably linked to a promoter element comprising one or a multiplicity of tandemly-repeated sequences that bind to the DNA-binding protein and are linked to at least a core promoter from a mammalian cellular or viral gene whose expression is induced by p21. In preferred embodiments, the fusion protein comprises a DNA binding protein that is yeast Gal4 or bacterial LexA or a sequence specific DNA binding fragment thereof. In preferred embodiments, the reporter gene is firefly luciferase, Renilla luciferase, chloramphenicol acetyltransferase, beta-galactosidase, green fluorescent protein, or alkaline phosphatase.

In particularly preferred embodiments, the mammalian cell comprises a first recombinant expression construct having a core promoter, wherein the core promoter comprises a sequence from about −46 to about +17 of a promoter from a cellular or viral gene whose expression is induced by p21. In preferred embodiments, the promoter is from connective tissue growth factor (SEQ ID NO. 1), adenovirus E1B promoter (SEQ ID NO. 2), adenovirus major late promoter (SEQ ID NO. 3), complement C3 (SEQ ID NO. 4), plasminogen activator inhibitor-1 (SEQ ID NO. 5), serum amyloid A (SEQ ID NO. 6), manganese superoxide dismutase (SEQ ID NO. 7), or herpes simplex virus thymidine kinase (SEQ ID NO. 8).

In additional preferred embodiments, the mammalian cell comprises yet another recombinant expression construct encoding p21, more preferably an inducible p21 gene. In alternative embodiments, the p21 encoded by said recombinant expression construct contains at least one mutation in its cyclin or cyclin-dependent kinase binding sites, wherein said mutations render p21 incapable of inhibiting cyclin/cyclin-dependent kinase complexes. Preferably, expression of p21 from the recombinant expression construct is mediated by contacting the cell with an inducing agent that induces transcription from the inducible promoter or by removing an agent that inhibits transcription from such a promoter.

In a second aspect, the invention provides a system for screening compounds that inhibit p21-mediated induction of gene expression. One embodiment of the screening systems of the invention comprises the first aspect of the invention disclosed above. In another embodiment, the system comprises a first cell comprising two recombinant expression constructs: a first recombinant expression construct that encodes a fusion protein between a sequence-specific DNA-binding protein and p300 or CRB or a truncated version thereof that maintains transcription activation activity and comprises a CRD1 amino acid sequence motif; and a second recombinant expression construct encoding a reporter gene operably linked to a promoter element comprising one or a multiplicity of tandemly-repeated sequences that bind to the DNA-binding protein and are linked to at least a core promoter from a mammalian cellular or viral gene whose expression is induced by p21. The system also comprises a second cell, that differs from the first cell because the promoter in the second recombinant expression construct in said second cell comprises a core promoter from a gene whose expression is not induced by p21 or that is mutated so that the promoter is unresponsive to p21. In alternative embodiments, the system comprises a first cell comprising a recombinant expression construct having a reporter gene operably linked to a complete promoter from a cellular or viral gene whose expression is induced by p21, and further comprising a second cell, which differs from the first cell by comprising a recombinant expression construct having a reporter gene operably linked to a complete promoter from a cellular or viral gene whose expression is induced by p21, wherein the promoter sequence is mutated so that the promoter is unresponsive to p21. In preferred embodiments, the promoter comprising the first recombinant expression construct of this aspect of the invention is a wild-type, p21-responsive promoter from Serum Amyloid A (SEQ ID NO. 13), and the promoter of the recombinant expression construct in the second cell is a mutated, p21-nonresponsive promoter from Serum Amyloid A (SEQ ID NO. 14).

In additional preferred embodiments, the mammalian cell comprises yet another recombinant expression construct encoding p21, more preferably an inducible p21 gene. In alternative embodiments, the p21 encoded by said recombinant expression construct contains at least one mutation in its cyclin or cyclin-dependent kinase binding sites, wherein said mutations render p21 incapable of inhibiting cyclin/cyclin-dependent kinase complexes. Preferably, expression of p21 from the recombinant expression construct is mediated by contacting the cell with an inducing agent that induces transcription from the inducible promoter or by removing an agent that inhibits transcription from such a promoter.

In a third aspect, the invention provides methods for identifying a compound that inhibits induction of gene expression by p21. In a preferred embodiment, the method comprises the steps of: culturing under conditions where p21 is induced a recombinant mammalian cell as described above in the first aspect of the invention in the presence or absence of a compound; comparing reporter gene expression in said cell in the presence of the compound with reporter gene expression in said cell in the absence of the compound; and identifying the compound that inhibits induction of gene expression by p21 if reporter gene expression in the presence of p21 is lower in the presence of the compound than in the absence of the compound. In alternative preferred embodiments, the method comprises the step of culturing in the presence and absence of the compound a first and a second cell of a system of the invention as described in the second aspect of the invention herein; comparing reporter gene expression in the first and the second cells in the presence of the compound with reporter gene expression in said cells in the absence of the compound; and identifying the compound that inhibits induction of gene expression by p21 if reporter gene expression is decreased in the presence of the compound in the first cell to a greater degree than in the second cell. In preferred embodiments, expression of the reporter gene is detected using an immunological reagent, by assaying for an activity of the reporter gene product, or by hybridization to a complementary nucleic acid.

In a fourth aspect, the invention provides compounds that inhibit p21-mediated induction of cellular or viral gene expression, wherein said compounds are identified by the methods of the invention.

In a fifth aspect, the invention provides methods for inhibiting p21-mediated induction of cellular or viral gene expression, comprising the step of contacting a cell with a compound identified according to the methods of the invention. In preferred embodiments, the compound is an antiviral compound.

In a sixth aspect, the invention provides methods of inhibiting or preventing expression of a gene induced by p21 in a mammalian cell. In this aspect, the methods comprise the step of contacting the mammalian cell with an amount of a compound identified by the methods of this invention effective to inhibit or prevent expression of a gene induced by p21. In a preferred embodiment, the methods are provided for treating an animal to prevent or ameliorate the effects of a disease associated with p21-induced gene expression. In this aspect, the methods comprise the step of administering to an animal in need thereof a therapeutically-effective dose of a pharmaceutical composition of a compound identified by the methods of this invention. In preferred embodiments, the animal is a human. In a preferred embodiment, the method is a method for having an antiviral effect on a mammalian cell, preferably wherein the animal is a human.

Specific preferred embodiments of the present invention will become evident from the following more detailed description of certain preferred embodiments and the claims.

### BRIEF DESCRIPTION OF THE DRAWINGS

FIGS. 1A though 1C represent experimental results showing that the level of p21 inducibility is dependent on the core promoter. FIG. 1A is a graph of luciferase activity in U-2 OS cells into which expression plasmids encoding Gal4 alone or Gal4 p300^{CRD1+ (}192-1044) were cotransfected with reporter plasmids containing the indicated core promoters. The absolute levels of luciferase activity and fold induction by Gal4 p300^{CRD1+} (192-1044) are shown. FIG. 1B is a histogram of reporter gene expression in the presence or absence of p21 expression. An RSV p21 expression plasmid, or appropriate RSV control, was cotransfected into U-2 OS cells with Gal4 p300^{CRD1+ (}192-1044) and reporter plasmids containing the indicated core promoters. Activation of transcription was calculated relative to the level seen with Gal4 alone. Results are expressed as fold inducibility by p21 (the ratio of luciferase activity seen in RSV control versus RSV p21 transfected cells). FIG. 1C shows an alignment of the TATA box regions of the promoters used herein, grouped according to their p21 inducibility. Regions of homology are underlined. Where no TATA box was present, the corresponding region of the promoter relative to the start site of transcription is shown.

FIGS. 2A and 2B show the results of experiments demonstrating that p21 inducibility of the AdML promoter is CRD1 dependent. FIG. 2A is a comparison of the ability of Gal4 p300^{CRD1+ (}192-1044) versus Gal4 p300^{CRD1-} (192-1004) (CRD-) to respond to p21 with the AdML and Bax core promoters. 40 fold less of the CRD-plasmid was used (0.25 ng) to compensate for its much higher level of transactivation. FIG. 2B is substantially the same experiment as shown in FIG. 2A, but Gal4 is fused with full-length p300 and shows the same promoter specificity seen with Gal4 p300^{CRD1+ (}192-1044). Results are expressed as the relative level of luciferase activity seen with the Gal4 p300 fusion to the level seen with Gal4 alone.

FIGS. 3A through 3D demonstrate that the AdML TATA box confers p21 inducibility on the core promoter. FIG. 3A is an alignment of the sequences of the hybrid core promoters. The Bax sequence is shown in italics, the TATA sequence is in bold and the start site of transcription of the wild type promoters are bold underlined. Heterologous promoter regions in the hybrids are underlined. Some hybrid promoters are of differing lengths due to differences in the spacing between the AdML and Bax TATA boxes and initiator elements. FIGS. 3B and 3C represent an analysis of the p21 inducibility of the hybrid promoters using Gal4 p300^{CRD1+ (}192-1044). Results are expressed as the relative level of luciferase activity seen with the Gal4 p300 fusion to the level seen with Gal4 alone. FIG. 3D shows that the absence of p21 inducibility is not a result of an inability of CRD1 to repress transcription. The results shown are from an analysis of the ability of Gal4 p300^{CRD1+} (192-1044) and Gal4 p300^{CRD1-} (192-1004) to stimulate transcription from the indicated hybrid promoters. Here, equivalent levels (5 ng) of both Gal4 expression plasmids are used.

FIGS. 4A through 4C demonstrate that sequences flanking both sides of the TATA box are required for p21 inducibility. FIG. 4A shows an alignment of the hybrid promoters containing different TATA box flanking sequences inserted into Bax. FIG. 4B was an expression plasmids encoding Gal4 alone or Gal4 p300^{CRD1+} (192-1044) were cotransfected with reporter plasmids containing the indicated core promoters. The absolute levels of luciferase activity and fold induction by Gal4 p300^{CRD1+} (192-1044) are shown. FIG. 4C shows an analysis of p21 inducibility of the hybrid promoters using Gal4 p300^{CRD1+} (192-1044). Results are expressed as the relative level of luciferase activity seen with the Gal4 p300 fusion to the level seen with Gal4 alone.

FIGS. 5A and 5B demonstrate that TBP/TFIIB binding does not correlate with p21 inducibility. The ³²P labeled probes and protein samples used in the EMSA assays are indicated in these Figures. FIG. 5A shows the results of EMSA analysis, demonstrating that the TBP/TFIIB complex binds the AdML TATA box but not the Bax TATA box. FIG. 5B shows that TBP/TFIIB binding is dependent upon the 3′ TATA flanking sequence but not the 5′ flanking sequence and therefore does not correlate with p21 inducibility. EMSA analysis was performed using the indicated ³²P labeled probes and the indicated protein samples.

FIG. 6 shows that p21 inducibility is also determined by the factors binding the upstream promoter, and can vary depending on the transactivation domain. The ability of Gal4 ER(AF2) and Gal4 p53 to be induced by cotransfection of RSV p21 was analyzed using the indicated reporter plasmids. Since Gal4 p53 is a strong activator of these reporter plasmids, only 50 pg of expression plasmid was used compared to 5 ng of Gal4 ER (AF2). Results are expressed as the relative level of luciferase activity seen with the Gal4 fusion to the level seen with Gal4 alone.

FIGS. 7A and 7B show the results of p21 induction assays using constructs containing a full-length serum amyloid A protein promoter. FIG. 7A shows that alteration of the TATA box in the serum amyloid A promoter reduces p21 inducibility. HT1080 p21-9 cells (a derivative of the HT1080 human fibrosarcoma cell line containing an IPTG inducible p21 gene) were transfected with either pGL3 SAA or pGL3 SAA (BAX TATA). 72 hours following IPTG induction of p21, cells were harvested and luciferase assays were performed. FIG. 7B shows that alteration of the TATA box in the Bax promoter does not confer p21 inducibility. HT1080 p21-9 cells were transfected with either pGL3 Bax or pGL3 Bax (ML TATA). 72 hours following IPTG induction of p21, cells were harvested and luciferase assays were performed.

FIGS. 8A through 8D shows the results of experiments demonstrating that p21 mediated regulation of CRD1 function is independent of Cyclin/CDK inhibition. FIG. 8A is a schematic diagram of p21 and the p21 Δ21,24 and p21 Δ53-58 cDNAs used in the experiments described in Example 10. FIG. 8B is a histogram showing that both p21 Δ21,24 and p21 Δ53-58 derepress the CRD1 domain. Expression plasmids encoding Gal4 alone (16.7 ng), Gal4 p300^{CRD1-} (192-1004) (1 ng) or Gal4 p300^{CRD+} (192-1044) (16.7 ng) were cotransfected as indicated with the Gal4 E1B reporter plasmid (1.67 μg) and RSV expression plasmids (1.67 μg) containing wild type p21, p21 Δ21,24 or p21 Δ53-58 into U-2 OS cells. The absolute levels of luciferase activity are shown. FIG. 8C is an autoradiogram showing that wild type p21 but not p21 Δ21,24 or p21 Δ53-58 induces dephosphorylation of Rb. 293 cells were transfected with 5 μg of the indicated RSV p21 expression plasmids. After 48 hours whole cell lysates were prepared and immunoblotted with an anti-Rb antibody. FIG. 8D is a histogram showing that wild type p21 but not p21 Δ21,24 and p21 Δ53-58 repress the Cyclin E promoter. U2-OS cells were transfected with Cyclin E-luciferase (1.67 μg) and the indicated RSV p21 expression plasmids (1.67 μg). The absolute levels of luciferase activity are shown.

### DETAILED DESCRIPTION OF THE PREFERRED EMBODIMENTS

The invention provides methods and reagents for identifying compounds that inhibit p21-mediated transcription induction of cellular and viral genes. The invention also provides methods for using said compounds to inhibit p21-mediated cellular or viral gene expression induction, methods for treating or preventing viral disease, and methods for treating or preventing pathogenic consequences of senescence and aging mediated by p21-induced gene expression.

For the purposes of this invention, reference to “a cell” or “cells” is intended to be equivalent, and particularly encompasses in vitro cultures of mammalian cells grown and maintained as known in the art. In preferred embodiments, the cells are mammalian cells, more preferably human cells and particularly human U2-OS osteosarcoma cells.

For the purposes of this invention, reference to “cellular genes” in the plural is intended to encompass a single gene as well as two or more genes. It will also be understood by those with skill in the art that effects of modulation of cellular gene expression, or reporter constructs under the transcriptional control of promoters derived from cellular genes, can be detected in a first gene and then the effect replicated by testing a second or any number of additional genes or reporter gene constructs. Alternatively, expression of two or more genes or reporter gene constructs can be assayed simultaneously within the scope of this invention.

For the purposes of this invention, the term “core promoter sequence” is intended to encompass a promoter sequence which is required for the initiation of transcription and which comprises a region from about −46 to about +17 as measured from the transcription initiation site in the promoter.

For the purposes of this invention, the term “pathological consequences of senescence and aging” is intended to encompass diseases such as cancer, atherosclerosis, Alzheimer's disease, amyloidosis, renal disease and arthritis.

For the purposes of this invention, the term “senescence” will be understood to include permanent cessation of DNA replication and cell growth not reversible by growth factors, such as occurs at the end of the proliferative lifespan of normal cells or in normal or tumor cells in response to cytotoxic drugs, DNA damage or other cellular insult.

The reagents of the present invention include any mammalian cell, preferably a rodent or primate cell, more preferably a mouse cell and most preferably a human cell, that can induce expression of p21, wherein such gene is either the endogenous gene or an exogenous gene introduced by genetic engineering.

In preferred embodiments, the invention provides mammalian cells containing a recombinant expression construct encoding a mammalian p21 gene. In preferred embodiments, the p21 gene is human p21 having nucleotide and amino acid sequences as set forth in U.S. Pat. No. 5,424,400, incorporated by reference herein. In alternative embodiments, the p21 gene is an amino-terminal portion of the human p21 gene, preferably comprising amino acid residues 1 through 78 of the native human p21 protein (as disclosed in U.S. Pat. No. 5,807,692, incorporated by reference) and more preferably comprising the CDK binding domain comprising amino acids 21-71 of the native human p21 protein (Nakanishi et al., 1995, EMBO J. 14: 555-563). Preferred host cells include mammalian cells, preferably rodent or primate cells, and more preferably mouse or human cells.

Recombinant expression constructs can be introduced into appropriate mammalian cells as understood by those with skill in the art. Preferred embodiments of said constructs are produced in transmissible vectors, more preferably viral vectors and most preferably retrovirus vectors, adenovirus vectors, adeno-associated virus vectors, and vaccinia virus vectors, as known in the art. See, generally, MOLECULAR VIROLOGY: A PRACTICAL APPROACH, (Davison & Elliott, ed.), Oxford University Press: New York, 1993.

In additionally preferred embodiments, the recombinant cells of the invention contain a construct encoding an inducible p21 gene, wherein the gene is under the transcriptional control of an inducible promoter. In more preferred embodiments, the inducible promoter is responsive to a trans-acting factor whose effects can be modulated by an inducing agent. The inducing agent can be any factor that can be manipulated experimentally, including temperature and most preferably the presence or absence of an inducing agent. Preferably, the inducing agent is a chemical compound, most preferably a physiologically-neutral compound that is specific for the trans-acting factor. In the use of constructs comprising inducible promoters as disclosed herein, expression of p21 from the recombinant expression construct is mediated by contacting the recombinant cell with an inducing agent that induces transcription from the inducible promoter or by removing an agent that inhibits transcription from such promoter. In preferred embodiments of this aspect of the inventive methods, the CDK inhibitor is p21. A variety of inducible promoters and cognate trans-acting factors are known in the prior art, including heat shock promoters than can be activated by increasing the temperature of the cell culture, and more preferably promoter/factor pairs such as the tet promoter and its cognate tet repressor and fusions thereof with mammalian transcription factors (as are disclosed in U.S. Pat. Nos. 5,654,168, 5,851,796, and 5,968,773), and the bacterial lac promoter of the lactose operon and its cognate lacI repressor protein. In a preferred embodiment, the recombinant cell expresses the lacI repressor protein and a recombinant expression construct encoding human p21 under the control of a promoter comprising one or a multiplicity of lac-responsive elements, wherein expression of p21 can be induced by contacting the cells with the physiologically-neutral inducing agent, isopropylthio-β-galactoside. In this preferred embodiment, the lacI repressor is encoded by a recombinant expression construct identified as 3′SS (commercially available from Stratagene, LaJolla, Calif.).

The invention also provides recombinant expression constructs wherein a reporter gene is under the transcriptional control of a promoter of a gene whose expression is modulated by p21, particularly genes whose expression is induced by p21. Preferred reporter genes comprising the second recombinant expression constructs of the invention include firefly luciferase, Renilla luciferase, chloramphenicol acetyltransferase, beta-galactosidase, green fluorescent protein, or alkaline phosphatase.

These reporter genes are then used as sensitive and convenient indicators of the effects of p21 gene expression, and enable compounds that inhibit the effects of p21 expression in mammalian cells to be easily identified. Host cells for these constructs include any cell in which p21 gene expression can be induced, and preferably include cells also containing recombinant expression constructs containing an inducible p21 gene as described above. Particularly preferred embodiments are human osteosarcoma cell line, U-2 OS.

In preferred embodiments, cells according to the invention comprise a first recombinant expression construct encoding a fusion protein between a sequence-specific DNA-binding protein and p300 or CRB or a truncated version thereof that maintains transcription activation activity and comprises a CRD1 amino acid sequence motif. In these embodiments, the sequence-specific DNA binding protein recognizes and binds to a particular sequence, most preferably a promoter sequence or a sequence contained in or found in or adjacent to a promoter, and which specifically binds to said sequence in a mammalian cell. In preferred embodiments, the sequence-specific DNA binding protein is yeast Gal4 protein, or a sequence-specific DNA binding portion or fragment thereof. In alternative embodiments, the sequence-specific DNA binding protein is bacterial lexA protein, or a sequence-specific DNA binding portion or fragment thereof (Fashena et al., 2000, Methods Enzymol. 328:14-26). Other sequence-specific DNA binding proteins that may be used include Lambda repressor (lambda cI) (Serebriskii et al., 1999, J Biol Chem 274:17080-17087). In preferred embodiments, this protein is fused with histone acetyltransferases p300 or CRB (cAMP responsive element binding protein) or a fragment thereof containing the CRD1 motif (Snowden et al., 2000, Id.).

In additional preferred embodiments, the mammalian cells of the invention also comprise a second recombinant expression construct encoding a reporter gene operably linked to a promoter element comprising one or a multiplicity of tandemly-repeated sequences that bind to a sequence-specific DNA-binding protein and are linked to at least a core promoter from a mammalian cellular or viral gene whose expression is induced by p21. As provided herein the promoter sequences that bind to a sequence-specific DNA-binding protein bind to the cognate sequence-specific DNA-binding protein encoded by the first recombinant expression construct. In a preferred embodiment, the promoter contains a single one of said sequences or more preferably 2, 3, 4, 5, or more tandemly-repeated sequences. These repeat sequences are linked to a core promoter from a mammalian cellular or viral gene whose expression is induced by p21. As used herein the term “core promoter” will be understood to mean that portion of the mammalian promoter extending at least from a position that is 46 nucleotides in the 5′ direction (and designated “−46”) to a position that is 17 nucleotides in the 3′ direction (and designated “+17”) from the transcription start site of the promoter. In preferred embodiments, the tandemly repeated sequences that bind to a sequence specific DNA binding protein are positioned 5′ to the start of the core promoter (−46), and the reporter gene is positioned 3′ to the end of the core promoter (+17). In preferred embodiments, the reporter gene is firefly luciferase, Renilla luciferase, chloramphenicol acetyltransferase, beta-galactosidase, green fluorescent protein, or alkaline phosphatase. In preferred embodiments, the core promoter is derived from a p21-inducible cellular or viral gene, as set forth in co-pending International Application, Publication No. WO 01/38532 (incorporated by reference). In additional preferred embodiments, the core promoter is a promoter derived from the connective tissue growth factor (SEQ ID NO. 1) promoter, adenovirus E1B promoter (SEQ ID NO.2), adenovirus major late promoter (SEQ ID NO. 3), complement C3 (SEQ ID NO. 4) promoter, plasminogen activator inhibitor-1 (SEQ ID NO. 5) promoter, serum amyloid A (SEQ ID NO. 6) promoter, manganese superoxide dismutase (SEQ ID NO. 7) promoter, or herpes simplex virus thymidine kinase (SEQ ID NO. 8) promoter. In preferred embodiments, the tandemly repeated sequence specifically binds to yeast Gal4 protein or a bacterial lexA protein or a sequence specific DNA binding site recognizing fragment thereof.

As provided by the invention, the mammalian cells comprising the first and second recombinant expression constructs are cells in which p21 expression can be induced. In certain embodiments, the induced p21 is the endogenous p21 encoded in the chromosomal DNA of the cell. In these embodiments, p21 gene expression can be induced, for example, by ionizing or ultraviolet radiation, by treatment with DNA-damaging or other cytotoxic drugs or with transforming growth factor β, by transduction with a vector encoding p53 that induces the transcription of p2 or (in the case of normal cells) by continuous passage in cell culture until the cells undergo replicative senescence.

In alternative preferred embodiments, the invention provides mammalian cells containing a recombinant expression construct encoding a-mammalian p21 gene. In preferred embodiments, the p21 gene is human p21 having nucleotide and amino acid sequences as set forth in U.S. Pat. No: 5,424,400, incorporated by reference herein. As shown in instant invention, the cyclin/CDK binding activity of p21 is not required for stimulation of the effect of p300/CBP. Therefore, in alternative embodiments the p21 gene contains mutations in any of its cyclin/CDK binding domains (described in Dotto, 2000, Biochem. Biophys. Acta 1471: M43-M56, incorporated herein by reference). More preferably, p21 mutants contain mutations in amino acids 21 and 24 (L21H and P24S), or a deletion from amino acids 53 to 58 (FVTETP deleted, replaced with PRG). Preferred host cells include mammalian cells, preferably rodent or primate cells, and more preferably mouse or human cells.

In additionally preferred embodiments, the recombinant cells of the invention contain a construct encoding an inducible p21 gene, wherein the gene is under the transcriptional control of an inducible promoter. In more preferred embodiments, the inducible promoter is responsive to a trans-acting factor whose effects can be modulated by an inducing agent. The inducing agent can be any factor that can be manipulated experimentally, including temperature and most preferably the presence or absence of an inducing agent. Preferably, the inducing agent is a chemical compound, most preferably a physiologically-neutral compound that is specific for the trans-acting factor. In the use of constructs comprising inducible promoters as disclosed herein, expression of p21 from the recombinant expression construct is mediated by contacting the recombinant cell with an inducing agent that induces transcription from the inducible promoter or by removing an agent that inhibits transcription from such promoter. A variety of inducible promoters and cognate trans-acting factors are known in the prior art, including heat shock promoters than can be activated by increasing the temperature of the cell culture, and more preferably promoter/factor pairs such as the tet promoter and its cognate tet repressor and fusions thereof with mammalian transcription factors (as are disclosed in U.S. Pat. Nos. 5,654,168, 5,851,796, and 5,968,773), and the bacterial lac promoter of the lactose operon and its cognate lacI repressor protein. In a preferred embodiment, the recombinant cell expresses the lacI repressor protein and a recombinant expression construct encoding human p21 under the control of a promoter comprising one or a multiplicity of lac-responsive elements, wherein expression of p21 can be induced by contacting the cells with the physiologically-neutral inducing agent, isopropylthio-β-galactoside. In this preferred embodiment, the lacI repressor is encoded by a recombinant expression construct identified as 3′SS (commercially available from Stratagene, LaJolla, Calif.).

The invention also provides a system for screening compounds that inhibit the induction of viral or cellular gene expression by p21. In this embodiment, the system comprises cells of the invention comprising a first recombinant expression construct encoding a fusion protein between a sequence-specific DNA-binding protein and p300 or CRB or a truncated version thereof that maintains transcription activation activity and comprises a CRD1 amino acid sequence motif, and a second recombinant expression construct encoding a reporter gene operably linked to a promoter element comprising one or a multiplicity of tandemly-repeated sequences that bind to a sequence-specific DNA-binding protein and linked to at least a core promoter from a mammalian cellular or viral gene whose expression is induced by p21. As provided herein the promoter sequences that bind to a sequence-specific DNA-binding protein bind to the cognate sequence-specific DNA-binding protein encoded by the first recombinant expression construct. The systems of the invention further comprise a second cell, which differs from the first cell by having a second recombinant expression construct in which the core promoter is from a gene whose expression is not induced by p21 or that is mutated so that the promoter is unresponsive to p21. The instant invention teaches how such mutations can be generated. Specifically, the core promoter sequences that are responsive to p21 preferably contain a TATA box flanked on the downstream side by an extended A/T rich sequence and more preferably are also flanked on the upstream side by a G/C rich region. Replacement of the extended TATA box sequences by a sequence that lacks such characteristics, including core promoter sequences from a p21-unresponsive gene, renders a promoter unresponsive or poorly responsive to p21.

In an alternative embodiment, the system comprises mammalian cells containing a recombinant expression construct having a reporter gene operably linked to a complete promoter from a cellular or viral gene whose expression is induced by p21, and a second cell, which differs from the first cell by having a recombinant expression construct comprising a complete promoter from a cellular or viral gene whose expression is induced by p21 operably linked to a reporter gene, but where the promoter sequence is mutated so that the promoter is unresponsive to p21. In preferred embodiments, the promoter is a promoter from a p21-inducible cellular or viral gene, as set forth in co-pending International Application, Publication No. WO 01/38532 (incorporated by reference). In particularly preferred embodiments, the promoter is serum amyloid A (SEQ ID NO. 13), and the mutations that render the promoter unresponsive to p21 are mutations of the extended TATA box (SEQ ID NO. 14), as described above.

In these embodiments, the cells of the system of the invention further advantageously comprise a recombinant expression construct encoding an inducible mammalian p21 gene. In preferred embodiments, the p21 gene is human p21 having nucleotide and amino acid sequences as set forth in U.S. Pat. No. 5,424,400, incorporated by reference herein. In alternative embodiments, the p21 gene contains mutations in any of its cyclin/CDK binding domains, more preferably mutations in amino acids 21 and 24 (L21H and P24S), or a deletion from amino acids 53 to 58 (FVTETP deleted, replaced with PRG).

In additionally preferred embodiments, the recombinant cells of the invention contain a construct encoding an inducible p21 gene, wherein the gene is under the transcriptional control of an inducible promoter. In more preferred embodiments, the inducible promoter is responsive to a trans-acting factor whose effects can be modulated by an inducing agent. The inducing agent can be any factor that can be manipulated experimentally, including temperature and most preferably the presence or absence of an inducing agent. Preferably, the inducing agent is a chemical compound, most preferably a physiologically-neutral compound that is specific for the trans-acting factor. In the use of constructs comprising inducible promoters as disclosed herein, expression of p21 from the recombinant expression construct is mediated by contacting the recombinant cell with an inducing agent that induces transcription from the inducible promoter or by removing an agent that inhibits transcription from such promoter. A variety of inducible promoters and cognate trans-acting factors are known in the prior art, including heat shock promoters than can be activated by increasing the temperature of the cell culture, and more preferably promoter/factor pairs such as the tet promoter and its cognate tet repressor and fusions thereof with mammalian transcription factors (as are disclosed in U.S. Pat. Nos. 5,654,168, 5,851,796, and 5,968,773), and the bacterial lac promoter of the lactose operon and its cognate lacI repressor protein. In a preferred embodiment, the recombinant cell expresses the lacI repressor protein and a recombinant expression construct encoding human p21 under the control of a promoter comprising one or a multiplicity of lac-responsive elements, wherein expression of p21 can be induced by contacting the cells with the physiologically-neutral inducing agent, isopropylthio-β-galactoside. In this preferred embodiment, the lacI repressor is encoded by a recombinant expression construct identified as 3′SS (commercially available from Stratagene, LaJolla, Calif.). Preferred host cells include mammalian cells, preferably rodent or primate cells, and more preferably mouse or human cells. In these embodiments, particularly preferred cells are fibrosarcoma cells, more preferably human fibrosarcoma cells and most preferably cells of the human HT1080 fibrosarcoma cell line and derivatives thereof. A most preferred cell line is an HT 1080 fibrosarcoma cell line derivative identified as HT1080 p21-9, deposited on Apr. 6, 2000 with the American Type Culture Collection, Manassas, Va. U.S.A. under Accession No. PTA 1664.

The invention also provides methods for identifying compounds that inhibit p21-mediated induction of cellular or viral gene expression. In this embodiment, the methods comprise the steps of culturing a recombinant mammalian cell according to the invention in the presence and absence of a compound and under conditions where p21 is induced. In certain preferred embodiments, the p21 that is induced in the recombinant mammalian cells is an endogenous p21 gene encoded in the cellular chromosomal DNA, and is induced by ionizing or ultraviolet radiation, by treatment with DNA-damaging or other cytotoxic drugs or with transforming growth factor β, by transduction with a vector encoding p53 that induces the transcription of p21 or (in the case of normal cells) by continuous passage in cell culture until the cells undergo replicative senescence. In alternative embodiments, the cell comprises a recombinant expression construct encoding p21 or mutant p21 containing mutations in any of its cyclin/CDK binding domains.

In further steps of these methods of the invention, expression of the reporter gene encoded by the recombinant expression constructs of the mammalian cells of the invention is compared in the cells cultured in the presence of the compound with expression in cells cultured in the absence of the compound. Compounds that inhibit p21-mediated induction of cellular or viral gene expression are identified if reporter gene expression is lower in the presence of the compound than in the absence of the compound. In preferred embodiments, reporter gene expression is assayed using an immunological detection reagent, or by the activity of the reporter gene product or using a nucleic acid that specifically hybridizes to reporter gene-encoding mRNA.

The invention provides alternative embodiments of methods for identifying a compound that inhibits induction of gene expression by p21. In these embodiments, the method comprises the steps of culturing the first and second cells of the systems of the invention in the presence and absence of a compound and under conditions where p21 is induced. In certain preferred embodiments, the p21 that is induced in the recombinant mammalian cells is an endogenous p21 gene encoded in the cellular chromosomal DNA, and is induced by ionizing or ultraviolet radiation, by treatment with DNA-damaging or other cytotoxic drugs or with transforming growth factor β, by transduction with a vector encoding p53 that induces the transcription of p21 or (in the case of normal cells) by continuous passage in cell culture until the cells undergo replicative senescence. In alternative embodiments, the cell comprises a recombinant expression construct encoding p21 or mutant p21 containing mutations in any of its cyclin/CDK binding domains.

In further steps of these embodiments of the inventive methods, reporter gene expression in the presence of p21 is compared between the first and second cells in the presence and absence of the compound. Compounds that inhibit p21-mediated induction of cellular or viral gene expression are identified if reporter gene expression is decreased in the presence of the compound in the first cell to a greater degree than in the second cell.

The invention also provides method for inhibiting p21-mediated induction of cellular or viral gene expression. In these embodiments, the methods comprises the step of contacting a cell with an effective amount of a compound identified according to the methods of the invention.

Also provided by the invention are compounds that inhibit p21-mediated induction of viral and cellular gene expression identified by the methods of the invention. In preferred embodiments, the compounds of the invention are antiviral compounds that inhibit expression of viral genes, most preferably viral genes from DNA viruses, most preferably double-stranded DNA viruses or viruses that have a double-stranded DNA portion of their lifecycle (such as retroviruses and most particularly lentiviruses such as human immunodeficiency virus). In other preferred embodiments, the compounds inhibit p21-mediated induction of expression of cellular genes associated with pathogenic consequences of senescence or aging, identified in International Application, Publication No. WO 01/38532 (incorporated by reference).

The invention also provides methods for treating an animal to prevent or ameliorate the effects of a disease accompanied by p21-induced gene expression. These methods comprise the step of administering to an animal in need thereof a therapeutically-effective dose of a pharmaceutical composition of a compound that inhibits p21-mediated gene expression induction identified according to the methods of the invention.

The invention also provides methods for inhibiting or preventing expression of a gene induced by p21 in a mammalian cell. These methods comprise step of contacting a mammalian cell with an amount of a compound identified according to the methods of the invention effective to inhibit or prevent expression of a gene induced by p21. These methods permit p21-induced genes to be selectively inhibited in an animal, most preferably a human.

The methods of the invention include methods for achieving an antiviral effect on a cell, comprising the step of contacting the cell with an effective amount of a compound that inhibits p21-mediated gene expression induction identified according to the methods of the invention.

The following Examples are intended to further illustrate certain preferred embodiments of the invention and are not limiting in nature.

### EXAMPLE 1

### Production of p21 Reporter Gene Constructs

A series of reporter plasmids were constructed to investigate whether p21 induction of viral and cellular gene expression was mediated by p300 transactivation as follows.

The reporter plasmids were based on the pGL3 Basic luciferase reporter plasmid (Promega, Madison, Wis.) backbone and had five Gal4 DNA-binding sites inserted upstream of the core promoter regions from a variety of p21 inducible and non-inducible genes. The pGL3 Basic plasmid was used because it lacks any eukaryotic promoter or enhancer sequences. The polylinker of pGL3 basic was removed by digestion with KpnI and HindIII and replaced by ligation to a double stranded oligonucleotide having the sequence:

GGTACTCGAGATCTCTAGAATCGAATTCAGCTT (SEQ ID NO. 15)

containing XhoI and EcoRI sites (underlined), with the KpnI and HindIII sites being destroyed (italics). Into the resulting construct was cloned the five Gal4 sites and E1B promoter from Gal4 E1B CAT (disclosed in Snowden et al., 2000, Mol. Cell. Biol. 20: 2676-2686, incorporated by reference) using the XhoI and EcoRI sites thereof. The E1B promoter, immediately downstream of the Gal4 sites, was then excised from this plasmid using XbaI and XmaI and replaced with double stranded oligonucleotides containing the core promoters for each of the plasmid constructs disclosed herein. All plasmid promoters were sequenced to confirm authenticity.

Core promoter sequences were obtained from Genbank or EPD (the Eukaryotic Promoter database, http://www.epd.isb-sib.ch/) and contained the sequences from +17 to −46 relative to the start site of transcription, where known. Where the start site was not known, the sequence was selected such that the positioning of core promoter elements was homologous to the other promoters described herein. These core promoters included five from human genes identified as being p21-inducible by cDNA microarray analysis and reverse transcription-PCR assays (as disclosed in co-pending International Application Publication No. WO 01/38532, incorporated by reference): complement C3 (Comp. C3), connective tissue growth factor (CTGF), plasminogen activator inhibitor 1 (PAI-1), serum amyloid A (SAA) and manganese superoxide dismutase (SOD2). Other promoters were derived from four human genes that are not induced by p21 according to the same assays (Bax (SEQ ID NO. 9), Cyclin D1 (SEQ ID NO. 10), Cyclin E (SEQ ID NO. 11) and p21 (SEQ ID NO. 12) itself and from three viral genes commonly used in studies on transcriptional regulation, adenovirus major late (AdML; SEQ ID NO. 3), adenovirus E1B (SEQ ID NO. 2) and herpes simplex virus thymidine kinase (HSV-TK; SEQ ID NO. 8).

Calcium phosphate transfections of U-2 OS cells were performed substantially as disclosed in Webster & Perkins (1999, Mol. Cell. Biol. 19: 3485-3495). Briefly, transfections were performed in 6 cm dishes using 2 μg of reporter plasmid, 1.6 μg of RSV p21 or RSV ADH control plasmid and 5 ng of Gal4 expression plasmid (except where indicated in the figure legend). Cells were harvested approximately 40 hours after transfection. Lysates were prepared using passive lysis buffer (Promega) and luciferase assays were performed according to manufacturer's instructions (Promega). All experiments were performed separately, a minimum of three times before calculating means and standard errors as shown in the Figures. Relative luciferase levels were calculated by referring to the level of activity seen with the Gal4 DNA-binding domain (amino acids 1-147) not fused to a heterologous activator, except when specifically referred to.

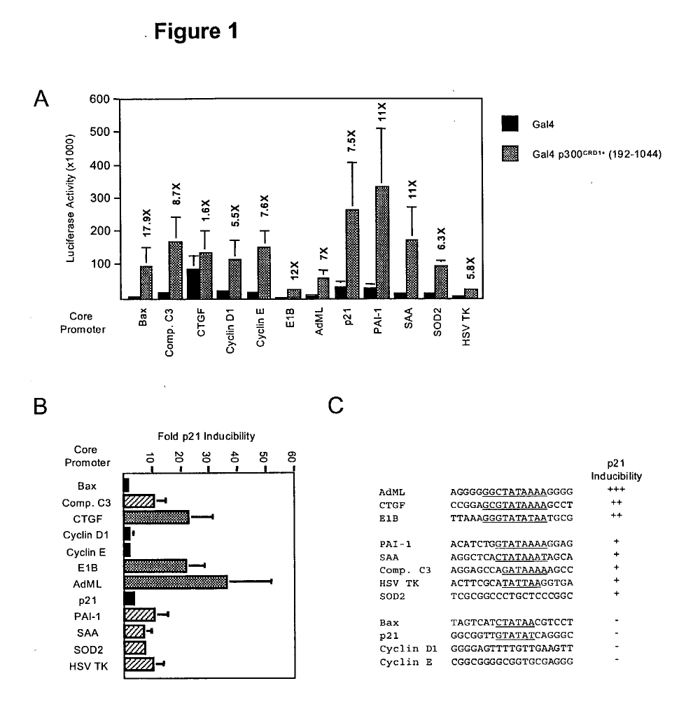

All of the reporter plasmids were transcriptionally active and were stimulated by co-transfection with an expression plasmid encoding a Gal4 fusion with amino acids 192-1044 of p300, which contains CRD1 (Gal4 p300^{CRD1+} (192-1044), shown in FIG. 1A); the Gal4-p300 fusion plasmid was prepared as described in Snowden et al. (2000, Id.). Some variability in reporter gene expression was seen, however. In particular, the Bax, E1B, AdML and HSV-TK core promoters conferred relatively low levels of basal activity although all were strongly activated by Gal4 p300^{CRD1+} (192-1044). In contrast, the CTGF, PAI-1 and p21 promoters had relatively high basal levels but, with the exception of CTGF, were still strongly activated by Gal4 p300^{CRD1+} (192-1044).

The p21 responsiveness of these plasmids was then determined by cotransfecting them into U-2 OS cells with Gal4 p300^{CRD1+} (192-1044), together with an RSV p21 expression plasmid or appropriate RSV vector control. The RSV p21 construct was prepared as disclosed in Perkins et al. (1997, Science 275: 523-527, incorporated by reference). In these experiments, relative levels of luciferase activity were calculated with respect to the level seen with Gal4 alone to ensure that any effects reported result from the p300 fusion protein. Analysis of these promoters immediately revealed that there were widespread differences in the p21 inducibility conferred by the different core promoter elements (shown FIG. 1B). For convenience these results are expressed as fold inducibility by p21, relative to the levels of Gal4 p300^{CRD1+} (192-1044) alone. The results from this experiment could be broadly broken down into three groups. One group, consisting of the AdML, E1B and CTGF core promoters, were highly p21-inducible. A second group, with core promoters derived from Comp. C3, PAI-1, SAA, SOD2 and HSV-TK showed an intermediate level of p21 inducibility. Finally, a third group of core promoters, Bax, Cyclin D1, Cyclin E and p21 itself, demonstrated little or no p21 inducibility. No correlation was observed between p21 inducibility and the intrinsic level of activity of these promoters seen in FIG. 1A. Although CTGF was minimally activated by Gal4 p300^{CRD1+} (192-1044) alone at the levels used in this experiment (5 ng), it displayed a high level of p21 inducibility. The sequences of these promoters are aligned as shown in FIG. 1C.

Thus, all the promoters found to be p21 inducible in vivo were also found to be p21 inducible in this assay, indicating that the assay is appropriate, inter alia, for identifying compounds that inhibit p21 transcriptional activation of these genes.

### EXAMPLE 2

### Identification of p21 Responsive Elements in Reporter Gene Constructs

Alignment of the promoter sequences shown in FIG. 1C above revealed that the only significant similarity between the majority of p21-inducible core promoters lay in the TATA box region. Of the highly inducible promoters, each had a canonical TATAA box flanked on the upstream side by a G/C rich region and on the downstream side by an extended A/T rich sequence. Amongst the promoters showing intermediate levels of p21 induction, 4 out of 5 had TATA boxes, although those of the Comp. C3 and HSV-TK promoters were non-consensus. Similar to the highly inducible promoters, all these TATA boxes had extended A/T rich downstream regions but differed from the AdML, E1B and CTGF promoters in their upstream flanking regions. An exception to this was the SOD2 core promoter, which lacked any sequence that might be construed as corresponding to a TATA box, but was still p21 inducible. Of the low or non-inducible promoters, 2 out of 4 lacked a TATA box. Of the remaining two, Bax and p21, the TATA box region, although present, diverged considerably from the highly inducible promoters, lacking both the upstream G/C rich region and the downstream A/T rich sequence.

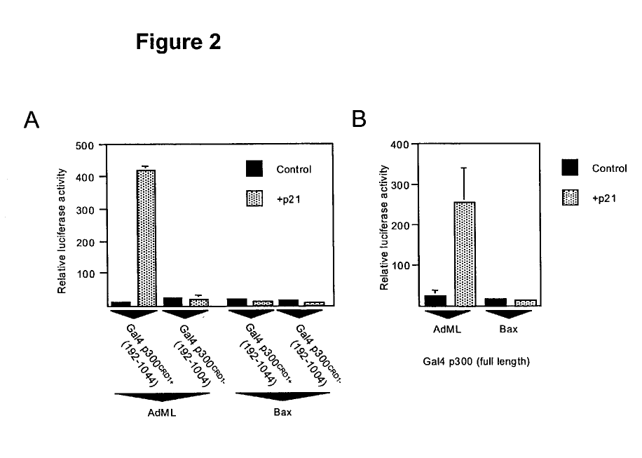

The two promoters, AdML and Bax, that showed the most divergent p21 response were selected to investigate these differences further. A reporter gene construct containing the AdML core promoter was co-transfected into U-2 OS cells with an expression plasmid encoding a Gal4 fusion with amino acids 192-1004 of p300, which lacks CRD1 (Gal4 p300^{CRD1-} (192-1004), prepared as disclosed in Snowden et al. (2000, ibid.), incorporated by reference). These experiments confirmed that p21 inducibility of AdML was dependent upon the CRD1 domain and does not result from an intrinsic effect on transcription from the AdML promoter (these results are shown in FIG. 2A). Since CRD1 is a potent repression domain, significantly lower levels of Gal4 p300^{CRD1-} (192-1004) were used in this experiment to allow the absence of p21 inducibility to be confirmed at levels of transcriptional activity seen with Gal4 p300^{CRD1+} (192-1044). No p21 inducibility is seen at higher levels of the Gal4 p300^{CRD1-} (192-1004) plasmid (data not shown). The difference in p21 inducibility between AdML and Bax was also seen with a Gal4 fusion of full length p300 (FIG. 2B). Because no difference was observed in p21 inducibility between Gal4 p300 (full length) and Gal4 p300^{CRD1+} (192-1044) (data not shown), subsequent experiments were performed with Gal4 p300^{CRD1+} (192-1044).

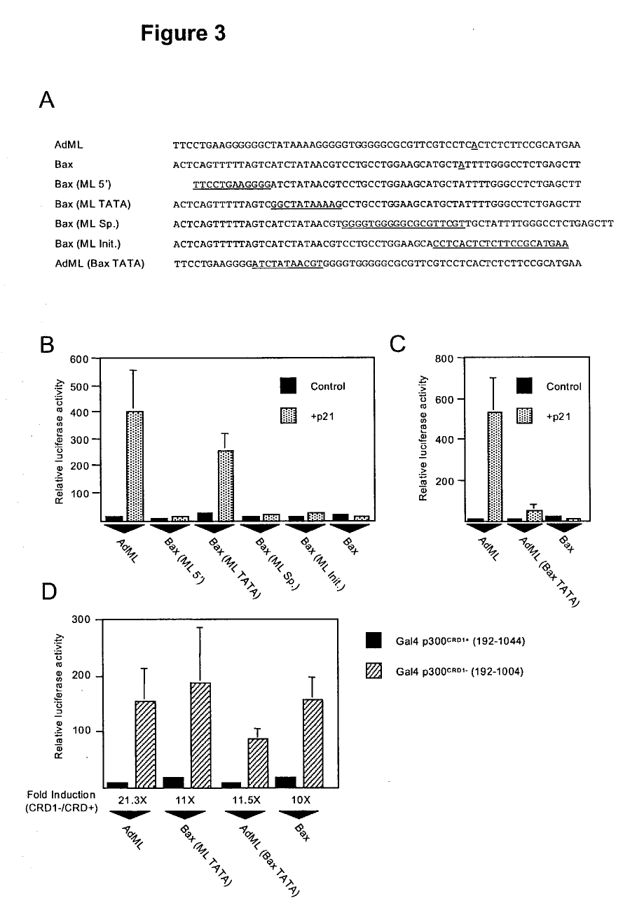

To determine the elements required for p21 inducibility or its absence, a series of plasmids were constructed where different sections of the AdML core promoter were transposed into their corresponding regions of the Bax promoter (shown in FIG. 3A). The pGL3 Bax and pGL3 Bax (ML TATA) constructs were prepared by PCR using the Bax luciferase reporter plasmid (Miyashita & Reed, 1995, Cell 80: 293-299) as a template. This plasmid was constructed using overlap extension PCR, where the 5′ and 3′ sections of the mutant promoter were generated first before a full length version (containing the region from −318 to +56 relative to the start site of transcription from the human Bax promoter) was created on a second round of PCR. The primers used were:

GGAGGTACCCGGGAATTCCAGACTGCAGTGAG (SEQ ID NO. 16) (5′ primer for both plasmids); and

CCTGAGCTCTCCCCAGCGCAGAAG (SEQ ID NO. 17) (3′ primer for both plasmids).

The mutant TATA box PCR primers were:

GTCGGCTATAAAAGCCTGCCTGGAAGCATGCTATTTTG (top strand) (SEQ ID NO. 18); and

CAGGCTTTTATAGCCGACTAAAAACTGAGTGGTTTTG (bottom strand) (SEQ ID NO. 19).

In the Figure, the sequence of the AdML TATA box introduced is underlined. The PCR amplification products were subcloned into pGL Basic using the KpnI and SacI sites present in the primer sequences. The identity of the constructs was confirmed by sequencing.

Analysis of these hybrid promoters demonstrated that only the 11 nucleotide region containing the TATA box was capable of conferring strong p21 inducibility on the Bax promoter (FIG. 3B). Confirming the importance of this element, transposition of the E1B TATA box region into Bax similarly conferred a high level of p21 inducibility (data not shown). In agreement with these results, replacement of the AdML TATA box region with that from Bax (FIG. 3A), virtually abolished p21 inducibility (FIG. 3C). Importantly, the absence of p21 inducibility with Bax and the hybrid promoters did not result from an inability of CRD1 to repress transcription, since deletion of this domain resulted in a similar increase in transcription from all promoters studied (FIG. 3D).

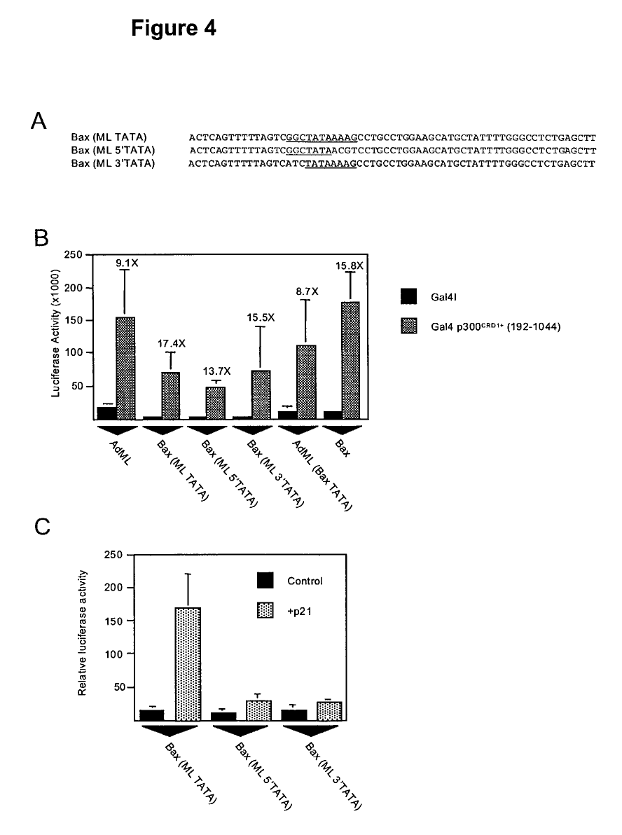

To determine the relative importance of both the upstream and downstream TATA flanking sequences, a further series of hybrid promoters were constructed (FIG. 4A). Swapping different core promoter elements has been shown to differentially affect both basal level and activated transcription in vitro (Wolner & Gralla, 2000, Mol. Cell. Biol. 20: 3608-3615). It was important, therefore, to compare the activity of these promoters with the parental vectors and other TATA box swap mutants. Although some differences in basal level of activity could be seen, all were transcriptionally active and were stimulated by Gal4 p300^{CRD1+} (192-1044) to approximately similar levels (FIG. 4B). Further analysis demonstrated that, although active, promoters with just the upstream or the downstream region from the AdML TATA box showed no significant level of p21 inducibility (FIG. 4C). p21 inducibility therefore depends on an extended sequence surrounding the TATA box.

The results set forth above indicated that the sequence surrounding the TATA box was involved in transcription induction by p21. The identification of these sequences and core promoters showing differential response to p21 provides an experimental approach to discriminating between compounds that specifically counteract the transcription-stimulating activity of p21, since such compounds are expected to have a stronger effect on the expression of p21-responsive promoters (in the presence of p21), relative to p21-unresponsive promoters.

The sequence of the TATA box and surrounding sequences was known in the art to influence binding of basal transcription factors (Lieberman et al., 1997, Mol. Cell. Biol. 17: 6624-6632). Since the Bax TATA sequence differs considerably from AdML, these constructs were used to determine whether the observed structural differences would be associated with differential binding of the core transcription factors TBP (TATA binding protein) and TFIIB. In these experiments, purified recombinant TBP and TFIIB were therefore incubated with ³²P labeled probes containing the AdML and Bax TATA boxes and analyzed by electrophoretic mobility shift assay (EMSA), performed as follows. ³²P labeled probe DNA was incubated for 1 hr at 30° C. with ˜1 ng purified recombinant TBP, ˜2 ng purified recombinant TFIIB (both proteins gifts from Dr Stefan Roberts, University of Manchester), 5 μg BSA, 250 ng poly(dG.dC) competitor and 12 μL buffer (having a formula of 10 mM Hepes pH7.9, 0.2 mM EDTA, 55 mM KCl, 4 mM MgCl₂, 5 mM ammonium sulphate, 8% v/v glycerol, 2% v/v polyethylene glycol, 5 mM β-mercaptoethanol, and 0.2 mM PMSF). Samples were then resolved using a 4.8% acrylamide gel (in 0.5×TB buffer). The gel was then dried and exposed to film.

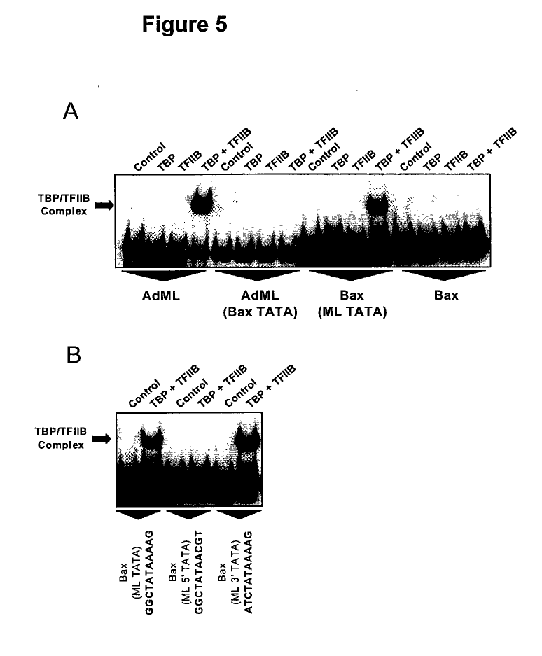

As expected (Evans et al., 2001, Genes Dev. 15: 2945-2949), a stable complex was only observed when both TBP and TFIIB were present (FIG. 3A) using the AdML TATA box construct. In contrast, no complex was seen with the Bax TATA box in this assay (FIG. 5A). Binding to Bax TATA box mutants containing either upstream or downstream sequences from AdML was also examined; neither of these mutants demonstrated p21 inducibility in the reporter gene assay (FIG. 4C). As expected, no binding was seen with the Bax (ML 5′ TATA) construct, where the extended A/T rich region typical of a consensus TATA box is missing (FIG. 5B). Significantly, Bax (ML 3′ TATA) bound TFIIB and TBP with apparently similar affinity to AdML TATA itself (FIG. 5B), despite not being p21 inducible (FIG. 4C). Thus, while strong binding of TBP and TFIIB might be a requirement for p21 inducibility, this result indicates that it is not the only factor involved.

Thus, these results indicate that p21 inducibility is mediated by sequences including the TATA box in these promoters, but that additional sequences in the TATA box region are also necessary for p21 inducibility.

To further investigate the physiological relevance of the findings set forth above, the capacity of p21 to regulate transactivation domains capable of recruiting endogenous p300 and CBP was investigated. In these studies, fusion constructs between Gal4 and the AF2 transactivation domain of the estrogen receptor (ER) or the amino terminal transactivation domain of the tumor suppressor p53 were prepared. Both of these transactivation domains were known in the art to interact with p300/CBP (Grossman, 2001, Eur. J. Biochem. 268: 2773-2778; Xu et al., 1999, Curr. Opin. Genet. Dev. 9:140-147); indeed, Gal4 p53 is repressed by the p300 CRD1 domain (Snowden et al., 2000, ibid.). Gal4 ER (AF2) was constructed by isolating a fragment encoding amino acids 280-555 from the human estrogen receptor alpha cDNA (provided by Dr. Simak Ali, Imperial College, London) using polymerase chain reaction. This fragment was then inserted into the EcoRI and BamHI sites of pCDNA3 Gal4 (Chapman & Perkins, 2000, J. Biol. Chem. 275: 4719-4725).

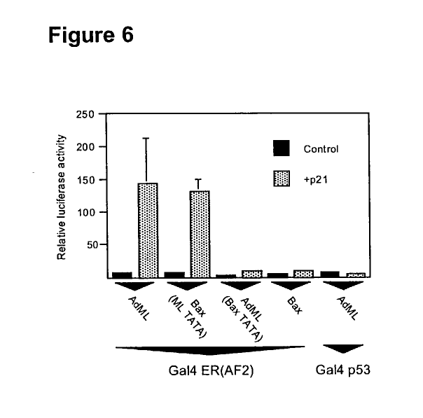

The results of these experiments are shown in FIG. 6. Significantly, Gal4 ER(AF2) transactivation was strongly stimulated by cotransfection of p21 with similar promoter specificity to that seen with Gal4 p300. Gal4 p53 activity, however, was completely unaffected by p21 co-transfection (FIG. 6).

Thus, these results indicated that the ability of p21 to induce transcription is decided not only by the nature of the TATA box but also by the type of activation domain recruited to the promoter.

### EXAMPLE 3

### Use of Reporter Gene Constructs in HT1080 Cells

These results set forth above indicated that p21 inducibility is a function both of the core promoter and upstream promoter elements. It was important therefore to identify whether the TATA sequence contributed to the ability of a full-length promoter to be p21 inducible. To facilitate this, a mutant was generated of the full length Serum Amyloid A (SAA) promoter, containing the region from −866 to −18, relative to the start site of transcription, in which the natural TATA box was replaced by that from the Bax promoter. This construct was prepared as follows. pGL3 SAA and pGL3 SAA (Bax TATA) reporter plasmids were constructed by PCR using Pwo Polymerase and the insert from pGL2 SAA as a template. Both plasmids contained the promoter region from −866 to −18 of the human serum amyloid A promoter relative to the start site of transcription, with pGL3 SAA (Bax TATA) having the natural TATA sequence replaced with that of the Bax promoter (ATCTATAACGT). The oligonucleotides used for the PCR were

GGCCTCGAGTGGCCACCATGCTCCTCCATAAGCC (5′ primer for both plasmids), (SEQ ID NO. 20);

GCCAGATCTCTGCTATTTATAGTGAGCCTTGCTGGTCTC (3′ primer for pGL3 SAA; SEQ ID NO. 21); and

GCCAGATCTCTGCACGTTATAGATAGCCTTGCTGGTCTC (3′ primer for pGL3 (Bax TATA); SEQ ID NO. 22)

The TATA sequences in both are underlined. The PCR products were subcloned into pGL3 basic using XhoI and BglII sites contained in the PCR primers. The plasmids were sequenced to confirm their identity.

Both wild type and mutant promoter-luciferase constructs were then transfected into HT1080 p21-9 cells (a derivative of the HT1080 human fibrosarcoma cell line containing an IPTG inducible p21 gene). These cells had previously been used to identify p21-regulated genes by microarray analysis, including SAA. (See co-pending International Application Publication No. WO 01/38532, incorporated by reference) These constructs were introduced into HT1080 cells using a modification of the transfection protocol set forth above. Transient transfection assays of full-length promoter-firefly luciferase constructs were carried out using a HT1080 p21-9 human fibrosarcoma cell line that expresses p21 from an isopropyl-β-thio-galactoside (IPTG)-inducible promoter (A.T.C.C. Accession No. PTA-1664). HT1080 p21-9 cells were grown in 15-cm tissue culture plates in DMEM supplemented with 10% FCII serum (Invitrogen) and then suspended in 400 μl of Opti-MEM medium (Invitrogen) at a concentration of 20-25 million cells per ml. 10 μg of the tested construct and 0.8 μg of a control plasmid pRL-CMV (Promega), which expresses Renilla luciferase from a CMV promoter, were added to the cells and transferred to a 0.4-cm gap electroporation cuvette (Bio-Rad). Cells were electroporated using Bio-Rad Gene Pulser and a capacitance extender (0.22 kV/960 μF) and plated in 12-well plates at 50,000 cells per well. Cells were cultured in the presence or in the absence of 50 μM IPTG for three days (in triplicates). Firefly and Renilla luciferase activities were measured using the Dual Luciferase Reporter Kit (Promega) according to the manufacturers instructions and using a Turner 20/20 single tube luminometer. The values for firefly luciferase activity were normalized to Renilla luciferase levels measured in the absence of IPTG.

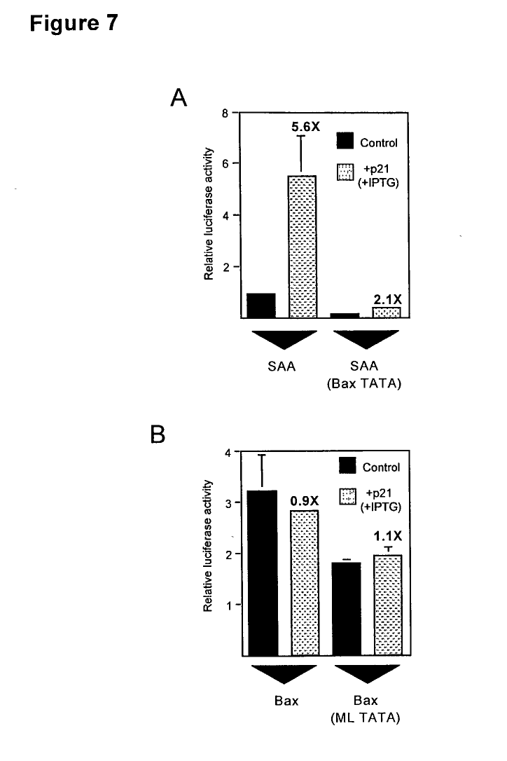

The results of these assays are shown in FIGS. 7A and 7B. As expected, the wild type SAA promoter was significantly induced upon p21 expression (FIG. 7A). In contrast, no induction was seen with the full-length wild type Bax promoter (containing nucleotides −318 to +56 relative to the start site of transcription) (FIG. 7B). Mutation of the SAA TATA box was seen to have two effects. Firstly, the basal level activity of the SAA promoter was reduced. Secondly, a significantly reduced level of p21 inducibility was seen, although this was not completely abolished (FIG. 5A). Preliminary analysis of the full-length complement C3 promoter has also demonstrated a similar, although less pronounced, reduction in p21 inducibility upon mutation of the TATA box (data not shown). A mutant full-length Bax promoter in which its TATA box had been replaced by that of AdML, did not become p21 inducible, however (FIG. 7B).

These results with full length SAA promoter are therefore consistent with an important role for the TATA box sequence and the factors that bind it in determining p21 inducibility in a full-length promoter context. These results also confirm, however, that the TATA box sequence alone is not sufficient for p21 inducibility and that an important contribution also comes from the factors binding the upstream promoter. Differential stimulation of the wild-type and mutated SAA promoters by p21 provides another experimental approach to discriminating between compounds that specifically counteract the effect of p21 in a full-length promoter assay, since such compounds are expected to have a stronger effect on the expression of the wild-type promoter of SAA or another p21-responsive gene, in the presence of p21, relative to the mutated promoter with diminished responsiveness to p21.

### EXAMPLE 4

### p21 Gene Expression Induction Independent of CDK Inhibition

Although p21 had previously been shown to derepress the activity of the CRD1 domains of p300 and CBP, it was unknown whether this was an indirect function of its ability to inhibit Cyclin/Cyclin dependent kinase (CDK) complexes (Snowden et al., 2000, ibid.). To determine whether CDK inhibition by p21 was or was not required for p21 gene transcription induction, two specific p21 mutants were utilized.

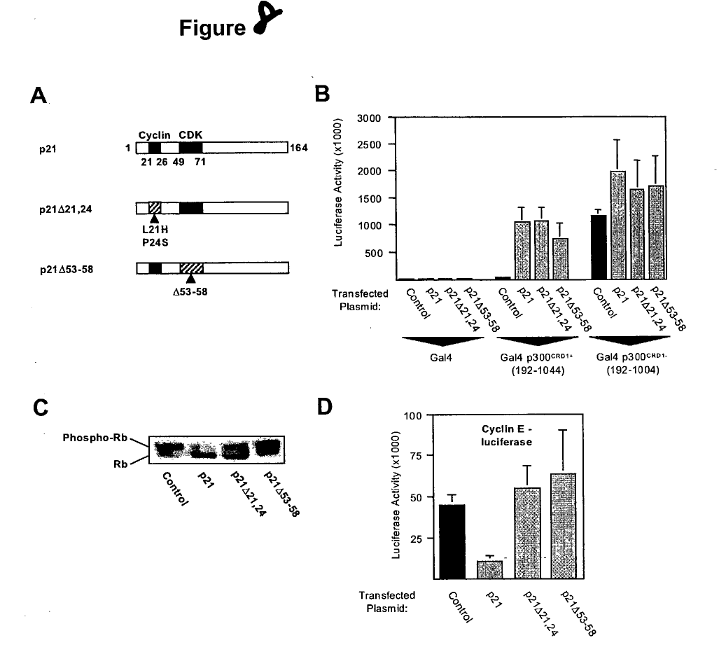

The RSV p21 expression plasmid has been described previously (Puri et al., 1997, ibid.). Mutant p21 cDNAs were obtained from Professor Nick LaThangue (University of Glasgow) and have been described previously and found to be inactive for Cyclin/CDK inhibition (Delavaine & La Thangue, 1999, Oncogene 18: 5381-5392). The mutant p21 plasmids (in pCDNA3) were subcloned into the same RSV expression plasmid as wild type p21 (pRc/RSV) using Hind III and XbaI. The first of these contained mutations in amino acids 21 and 24 (L21H and P24S), which inhibit Cyclin binding by p21. The second mutant contained a deletion extending from amino acids 53 to 58 (FVTETP deleted, replaced with PRG) which inhibits CDK binding (FIG. 8A).

Wild type p21 and the two mutants were cotransfected with Gal4 p300^{CRD+} (192-1044) and Gal4 E1B luciferase into U-2 OS cells. Both mutants stimulated transcriptional activity to the same extent as the wild type protein (FIG. 8B), consistent with a Cyclin/CDK independent role for p21 regulation of CRD1. Only a minimal effect of wild type p21 or the mutants was seen in co-transfections with Gal4 p300^{CRD1-} (192-1004), which lacks the CRD1 domain. To confirm that these mutations abolished Cyclin/CDK inhibition, expression plasmids encoding wild type p21 and the two p21 mutants were transfected into 293 cells, which have a very high transfection efficiency that allowed for whole population analysis. Immunoblot analysis of extracts prepared from these cells demonstrated that phosphorylation of the Rb tumor suppressor was inhibited by wild type p21, but no significant effect was seen with either mutant (FIG. 8C). Immunoblot analysis confirmed that both mutants were expressed at the same level as wild type p21 (data not shown). Furthermore, although wild type p21 could efficiently repress the activity of an E2F-dependent Cyclin E promoter-luciferase reporter plasmid in U-2 OS cells, neither p21 mutant was capable of doing so (FIG. 8D). These results confirmed that both mutations were compromised in their ability to inhibit Cyclin/CDK activity. In addition to these results, treatment with either mimosine, a rare plant amino acid that reversibly blocks the cell cycle at the G1/S phase boundary (Watson et al., 1991, Cytometry 12: 242-246) or roscovitine, an inhibitor of the Cdc2, Cdk2 and Cdk5 Cyclin dependent kinases (Meijer et al., 1991, Eur. J. Biochem. 243: 527-536), does not result in CRD1 dependent stimulation of transcription (data not shown). Taken together these results demonstrated that p21 regulates the CRD1 domain of p300 independently of cyclin dependent kinase inhibition.

These findings make it possible to distinguish compounds that specifically counteract the transcription-stimulating activity of p21 from compounds that counteract more general effects of p21, since only the former but not the latter compounds should be able to counteract the effect of p21 mutants that are deficient in cyclin/CDK inhibition.

It should be understood that the foregoing disclosure emphasizes certain specific embodiments of the invention and that all modifications or alternatives equivalent thereto are within the spirit and scope of the invention as set forth in the appended claims.

We claim:

1. A mammalian cell, wherein expression of p21 can be induced, comprising
(a) a first recombinant expression construct encoding a fusion protein between a sequence-specific DNA-binding protein and p300 or CRB or a truncated version thereof that maintains transcription activation activity and comprises a CRD1 amino acid sequence motif, and
(b) a second recombinant expression construct encoding a reporter gene operably linked to a promoter element comprising one or a multiplicity of tandemly-repeated sequences that bind to the DNA-binding protein of (a) and linked to at least a core promoter from a mammalian cellular or viral gene whose expression is induced by p21.

2. A mammalian cell according to claim 1, wherein the DNA-binding protein of subpart (a) is a yeast Gal4 protein or a bacterial lexA protein or a sequence specific DNA binding fragment thereof.

3. A mammalian cell according to claim 1, wherein the reporter gene of subpart (b) encodes firefly luciferase, Renilla luciferase, chloramphenicol acetyltransferase, beta-galactosidase, green fluorescent protein, or alkaline phosphatase.

4. A mammalian cell according to claim 1, wherein the core promoter of subpart (b) comprises a sequence from about −46 to about +17 of a promoter from a cellular or viral gene whose expression is induced by p21.

5. A cell according to claim 1, wherein the core promoter is from the connective tissue growth factor (SEQ ID NO. 1) promoter, adenovirus E1B promoter (SEQ ID NO. 2), adenovirus major late promoter (SEQ ID NO. 3), complement C3 (SEQ ID NO. 4) promoter, plasminogen activator inhibitor-1 (SEQ ID NO. 5) promoter, serum amyloid A (SEQ ID NO. 6) promoter, manganese superoxide dismutase (SEQ ID NO. 7) promoter, or herpes simplex virus thymidine kinase (SEQ ID NO. 8) promoter.

6. A mammalian cell according to claim 1, wherein expression of p21 is induced by treating the cell with agents that induce endogenous p21 gene in the cell.

7. A mammalian cell according to claim 1, further comprising a recombinant expression construct encoding p21.

8. A mammalian cell according to claim 7, wherein p21 is operably linked to an inducible promoter.

9. A mammalian cell according to claim 7 or claim 8, wherein p21 contains at least one mutation in its cyclin or cyclin-dependent kinase binding sites, wherein said mutations render p21 incapable of inhibiting cyclin/cyclin-dependent kinase complexes.

10. A mammalian cell according to claim 1, wherein such cell is U-2 OS osteosarcoma.

11. A mammalian cell according to claim 8, wherein expression of p21 from the recombinant expression construct is mediated by contacting the cell with an inducing agent that induces transcription from the inducible promoter or by removing an agent that inhibits transcription from such a promoter

12. A system for screening compounds that inhibit the induction of viral or cellular gene expression by p21, the system comprising cells according to claims 1-10, and further comprising a second cell, which differs from the cell of claims 1-10 in having a core promoter from a gene whose expression is not induced by p21 or that is mutated so that the promoter is unresponsive to p21.

13. A system for screening compounds that inhibit induction of gene expression by p21, such system comprising a first cell comprising a recombinant expression construct having a reporter gene operably linked to a promoter from a cellular or viral gene whose expression is induced by p21, and further comprising a second cell, which differs from the first cell by comprising a recombinant expression construct having a reporter gene operably linked to a promoter from a cellular or viral gene whose expression is induced by p21, wherein the promoter sequence is mutated so that the promoter is unresponsive to p21.

14. A system of claim 13, wherein the promoter of the recombinant expression construct of the first cell is a wild-type, p21-responsive promoter from Serum Amyloid A (SEQ ID NO. 13), and the promoter of the recombinant expression construct in the second cell is a mutated, p21-nonresponsive promoter from Serum Amyloid A.

15. A system of claim 13, wherein the mammalian cell is HT1080 fibrosarcoma

16. A system according to claim 13, wherein the first cell and the second cell further comprise a recombinant expression construct encoding p21.

17. A system according to claim 16, wherein p21 is operably linked to an inducible promoter in the recombinant expression construct comprising the first cell and the second cell.

18. A system according to claim 17, wherein expression of p21 from the recombinant expression construct is mediated by contacting the first and second cells with an inducing agent that induces transcription from the inducible promoter or by removing an agent that inhibits transcription from such a promoter.

19. A system according to claim 17, wherein each of the first and second cells further comprises a recombinant expression construct encoding a bacterial lactose repressor, wherein transcription thereof is controlled by a mammalian promoter, wherein the recombinant expression construct encoding p21 comprises a lactose repressor-responsive promoter element and wherein transcription of p21 is controlled by said lactose-repressor responsive promoter element, and wherein expression of p21 from the recombinant expression construct is mediated by contacting the cell with a lactose repressor-specific inducing agent

20. A mammalian cell according to claim 19, wherein the cell is a human HT1080 fibrosarcoma cell.

21. A mammalian cell of claim 20, identified by A.T.C.C. Accession No. PTA 1664 (HT1080 p21-9).

22. A mammalian cell according to claims 19 or 21, wherein the lactose repressor-specific inducing agent is a β-galactoside.

23. A method for identifying a compound that inhibits induction of gene expression by p21, the method comprising the steps of:
(a) culturing a recombinant mammalian cell according to claims 1-10 under conditions where p21 is induced in the presence and absence of a compound;
(b) comparing reporter gene expression in said cell in the presence of the compound with reporter gene expression in said cell in the absence of the compound; and
(c) identifying the compound that inhibits induction of gene expression by p21 if reporter gene expression is lower in the presence of the compound than in the absence of the compound.

24. A method for identifying a compound that inhibits induction of gene expression by p21, the method comprising the steps of:
(a) culturing the first and the second cell according to claims 12 or 13 under conditions where p21 is induced in the presence and absence of a compound;
(b) comparing reporter gene expression in the first and the second cells in the presence of the compound with reporter gene expression in said cells in the absence of the compound; and
(c) identifying the compound that inhibits induction of gene expression by p21 if reporter gene expression is decreased in the presence of the compound in the first cell to a greater degree than in the second cell.

25. The method of claim 23 or 24, wherein expression of the reporter gene is detected using an immunological reagent.

26. The method of claim 23 or 24, wherein expression of the reporter gene is detected by assaying for an activity of the reporter gene product.

27. The method of claim 23 or 24, where expression of the reporter gene is detected by hybridization to a complementary nucleic acid.

28. A method for inhibiting p21-mediated induction of cellular or viral gene expression, comprising the step of contacting a cell with a compound identified according to the method of claim 23 or 24.

29. A compound that inhibits expression of viral genes, or cellular genes associated with pathogenic consequences of senescence or aging, the compound produced by a method having the steps of:
(a) culturing a recombinant mammalian cell according to claims 1-10 under conditions where p21 is induced in the presence and absence of the compound;
(b) comparing reporter gene expression in said cell in the presence of the compound with reporter gene expression in said cell in the absence of the compound; and
(c) identifying the compound that inhibits induction of gene expression by p21 if reporter gene expression is lower in the presence of the compound than in the absence of the compound.

30. A compound that inhibits expression of viral genes, or cellular genes associated with pathogenic consequences of senescence or aging, the compound produced by a method having the steps of:
(a) culturing the first and the second cell according to claims 12 or 13 under conditions where p21 is induced in the presence and absence of a compound;
(b) comparing reporter gene expression in the first and the second cells in the presence of the compound with reporter gene expression in said cells in the absence of the compound; and
(c) identifying the compound that inhibits induction of gene expression by p21 if reporter gene expression is decreased in the presence of the compound in the first cell to a greater degree than in the second cell.

31. A compound according to claims 29 or 30 that is an antiviral compound.

32. A method for treating an animal to prevent or ameliorate the effects of a disease accompanied by p21-induced gene expression, the method comprising the steps of administering to an animal in need thereof a therapeutically-effective dose of a pharmaceutical composition of a compound according to 29 or 30.

33. A method for inhibiting or preventing expression of a gene induced by p21 in a mammalian cell, the method comprising the step of contacting the mammalian cell with an amount of a compound according to claim 29 or 30 effective to inhibit or prevent expression of the a gene induced by p21.

34. A method for achieving an antiviral effect on a cell comprising the step of contacting the cell with an effective amount of a compound according to claim 29 or 30.

35. A method for selectively inhibiting induction of genes by p21 in an animal, the method comprising the steps of administering to the animal a compound according to claim 29 or 30.

36. The method of claim 35, wherein the animal is a human.

37. A method for treating an animal to prevent or ameliorate the pathological consequences of senescence and aging associated with p21-induced gene expression, the method comprising the steps of administering to an animal in need thereof a therapeutically-effective dose of a pharmaceutical composition of a compound according to 29 or 30.

38. The method of claim 37, wherein the animal is a human.

39. The method of claim 37, wherein the pathological consequences of senescence and aging comprise cancer, atherosclerosis, Alzheimer's disease, amyloidosis, renal disease and arthritis.
